(19) United States
(12) Patent Application Publication
(10) Pub. No.: US 2018/0018429 A1
(43) Pub. Date: Jan. 18, 2018

(54) SYSTEM AND METHOD FOR COORDINATING PHYSICIAN MATCHING

(71) Applicant: Dignity Health, San Francisco, CA (US)

(72) Inventor: Adam Rice, Paradise Valley, AZ (US)

(21) Appl. No.: 15/547,861

(22) PCT Filed: Feb. 3, 2016
(86) PCT No.: PCT/US2016/016441

§ 371 Date: Aug. 1, 2017

Related U.S. Application Data
(60) Provisional application No. 62/111,529, filed on Feb. 3, 2015.

(51) Int. Cl. G06F 19/00; G06F 17/30; G06Q 10/10; G06Q 50/00

(52) U.S. Cl. CPC G06F 19/322; G06F 17/30867; G06F 19/327; G06F 19/363; G06F 17/30554; G06Q 10/109; G06Q 50/01; G06F 19/3406

(57) ABSTRACT

A system and method for coordinating physician matching for a user. The system including an interface configured to receive preference data related to the user. The system further includes a physician recommendation module being applied to the preference data and corresponding physician data. The physician recommendation module can track the preference data received by the interface, compare the preference data to the corresponding physician data, and recommend a list of physicians to the user when the physician data is above a threshold value. A display coupled to the recommendation module and configured to display the list of physicians to the user.

### CROSS-REFERENCE TO RELATED APPLICATIONS

This application claims priority to U.S. Provisional Patent Application No. 62/111,529, filed on Feb. 3, 2015, and entitled “SYSTEM AND METHOD FOR COORDINATING PHYSICIAN MATCHING.”

### STATEMENT REGARDING FEDERALLY SPONSORED RESEARCH

N/A

### BACKGROUND

The present disclosure relates to systems and methods for coordinating physician matching for a patient. More particularly, the disclosure relates to systems and methods for recommending one or more physicians to the patient based on patient preferences matching physician profiles.

The process of choosing a healthcare provider is currently time consuming and tedious. Patients may unsuccessfully meet and spend time with, multiple physicians until one is found that suits the patient's specific needs. A variety of factors exist that patients tend to consider when choosing a physician, and these factors are not uniform across all patients. Often, patients do not have enough information upon which to base a decision. Much of the information regarding physicians is not compiled in a format that consumers can access and use to make an informed decision. Additionally, the patient often has little to no input on what aspects of the data are important to him or her.

Further, across most major health care systems and payors, the most heavily trafficked section of the website (and top transaction) is the physician directory feature. However, performing a standard search for a suitable physician in the physician directory may often return an overwhelming number of results. Some results may not relate to medical practitioners. Other results may relate to practitioners that do not practice in a desired field or location. The imprecision of current search procedures cause wasted time and missed client-physician relationships. In addition, most major health systems and payors are using antiquated physician search tools, based on outdated search technologies and processes.

For example, most physician directories function as a utility to provide consumers with a large list of physicians within a geographic radius of a patient, that meet a limited set of search criteria (e.g., specialty, insurance accepted, accepting new patients, etc.). This leaves most patients overwhelmed with too many choices and typically requires the consumer to call each practice or conduct additional “background” searches on additional websites in order to determine if the physician is a good fit for them. Thus, a burden is placed on the patient to make a physician decision based on a limited set of information.

Thus, there is a need for systems and methods that utilize the physician directory tool to begin the consumer engagement process to provide a physician recommendation tool. There is also a need to combine multiple dimensions of user preferences and other data to dynamically create a recommended physician results list provided on an accessible interface for users to define preferences and receive search results.

### SUMMARY

The present disclosure overcomes the aforementioned drawbacks by providing a physician recommendation system that allows patients to input personal preferences and profile information into the system when searching for a new physician. Based on the patient's input data, a list of recommended physicians that may be compatible is provided to the patient. The physician directory platform may be integrated with online appointment scheduling, “virtual” visits, couponing/discount service, and ratings/reviews to help the patient consider their recommended physician. Thus, health care systems may more effectively deliver the appropriate care options to the patient in a single integrated experience, as opposed to multiple applications and software disparately displayed as stand-alone services for consumers.

In accordance with one aspect of the disclosure, a system for coordinating physician matching is disclosed. The system includes an interface configured to receive preference data related to the user. The system further includes a physician recommendation module being applied to the preference data and corresponding physician data. The physician recommendation module can track the preference data received by the interface, compare the preference data to the corresponding physician data, and recommend a list of physicians to the user when the physician data is above a threshold value. A display, coupled to the recommendation module, is configured to present the list of physicians to the user based on the most compatible (highest confidence) matches.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a system configured to implement the present disclosure.

FIG. 2 is a flow chart setting forth the steps of processes for generating, a physician recommendation list based on user preferences in accordance with the present disclosure.

FIG. 3 is a more detailed flow chart setting forth the steps of processes for generating a physician recommendation list based on user preferences in accordance with the present disclosure.

FIGS. 3A-3H are non-limiting example interfaces used in generating a physician recommendation list based on user preferences in accordance with the present disclosure.

FIGS. 4A-4D are non-limiting examples of direct matching, persona matching, health needs matching and disparate data matching used in generating a physician recommendation list based on user preferences in accordance with the present disclosure.

FIGS. 5A-5B are non-limiting examples of visual representation of a matching technique used in generating a physician recommendation list based on user preferences in accordance with the present disclosure.

FIG. 5C is a flow chart setting forth the steps of processes for generating a physician recommendation list based on user preferences in accordance with the present disclosure.

FIG. 6 is a flow chart setting forth the steps of processes for generating a physician recommendation list based on user preferences in accordance with the present disclosure.

FIGS. 6A-6B are non-limiting examples of interfaces used in generating a physician recommendation list based on user preferences in accordance with the present disclosure.

### DETAILED DESCRIPTION

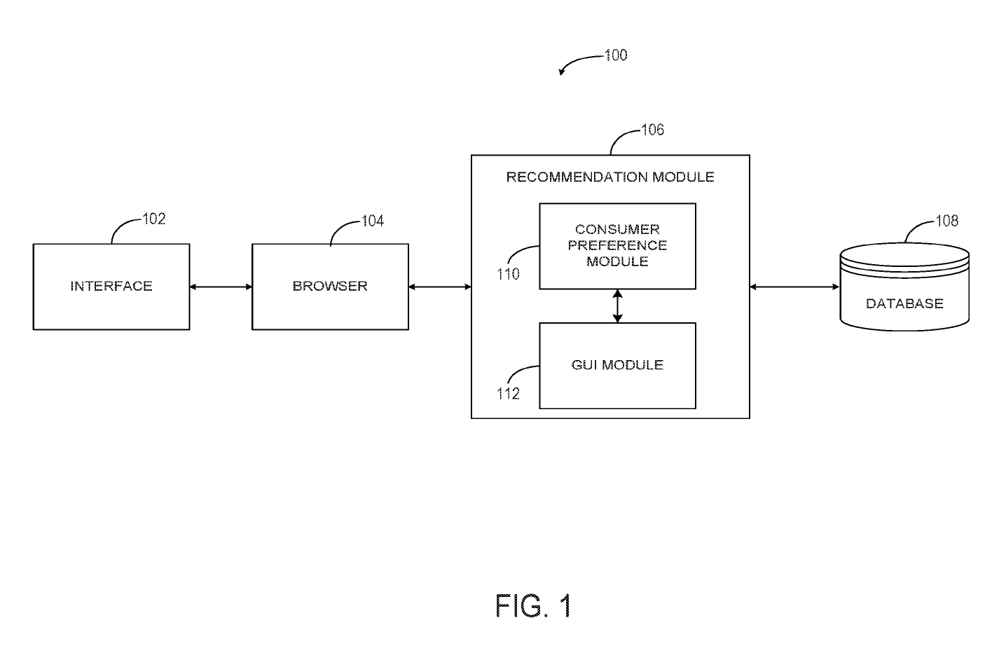

Referring particularly now to FIG. 1, a physician recommendation system 100 is shown that is configured to provide a list of recommended physicians to a patient based on the patient preferences. In general, the physician recommendation system 100 includes an interface 102 that is displayed by a browser 104, a recommendation module 106, and a database 108. The components of the physician recommendation system 100 may be located on the same device including, but not limited to, a server, mainframe, desktop Personal Computer (PC), laptop, Personal Digital Assistant (PDA), telephone, mobile phone, kiosk, cable box, and the like. Alternatively, the components of the physician recommendation system 100 may be located on separate devices connected by a network (e.g., the Internet), with wired and/or wireless segments.

In one non-limiting example, the physician recommendation system 100 may include multiple interfaces 102 that may be accessed and manipulated by multiple users simultaneously. Similarly, various instances of the interface 102 may be accessed on multiple browsers 104. Additionally, the physician recommendation system 100 may be optionally connected to multiple databases 108. The physician recommendation system 100 may connect to the databases 108 physically and/or over a network, for example. For purposes of this disclosure, the terms server or server computer may refer to any combination of the components of the physician recommendation system 100, running any of the algorithms for the software modules described herein (e.g., a server computer recommending one or more physicians using the recommendation module, rendering and transmitting a user interface on a web page using a server-side graphical user interface module, and displaying the interface on a client computer using a client-side graphical user interface module, as described below).

The browser 104 may be configured to display the interface 102, which may be a graphical user interface (GUI) associated with the physician recommendation system 100. Those skilled in the art, having benefit of this detailed description, will appreciate that there will be many ways in which a GUI may be viewed other than using a browser.

The recommendation module 106 may include a consumer preference module 110 and a GUI module 112. The recommendation module 106 may be configured to recommend a physician to a user based on existing user preferences and data. The GUI module 112 is configured to enable a GUI for display in the browser 104. The consumer preference module 110, in combination with the recommendation module 106, may be configured to create recommendations for physicians based on user preferences and data. The consumer preference module 110 may be configured to solicit preference and selection information from a patient which is used to generate a list of recommended physicians. The consumer preference module 110 may also retrieve information from the database 108 to present the patient with an initial list of physicians from which to select. The consumer preferences may be obtained by providing the patient with choices to personalize desired physician characteristics. In addition, the consumer preference module 110 may adjust the relative importance of different metrics or categories based on preferences obtained from the consumer using an algorithm, for example.

The server computer(s) may therefore execute the method steps within one or more of the disclosed algorithm by sending instructions, possibly in the form of compiled and executable software code for any of the disclosed software modules, to a processor on the server computer(s). The processor may then execute these instructions causing the server computer(s) to complete the disclosed′method steps.

The database 108 may be configured to store data associated with the physician recommendation system 100. The database 108 may be any sort of system capable of storing data, such as a relational database, a database management system, a hierarchical file, a flat file, and the like. The database 108 may be directly connected with the recommendation module 106, any of the disclosed software modules, and/or to the server computers themselves, through various network configurations (e.g., wired, wireless, LAN WAN, etc.). The database 108 may include data relating to one or more physicians, as well as patient preferences and profile data that may later be compared by the recommendation module 106 to identify one or more physicians for the patient.

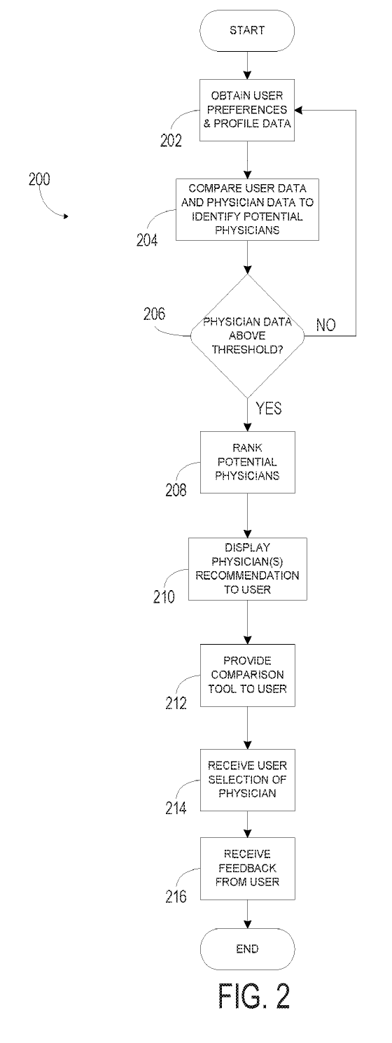

Referring now to FIG. 2, a flow chart setting forth exemplary steps 200 for generating a physician recommendation list based on matching user preferences to physician data is provided. To begin, user preferences and profile data may be obtained from the patient through the interface 102, displayed on a client computer, and sent to the consumer preference module 110 at process block 202. As noted above, the GUI module 112 may generate the user interface 102, possibly as a web page displayed on a browser 104, or, for example, as an app displayed on a smart phone or tablet. The user interfaces displayed throughout the drawings represent non-limiting examples of the user interfaces 102 that may be generated by the GUI module 112 and displayed on a client computer, such as a desktop computer, laptop computer, smart phone or tablet.

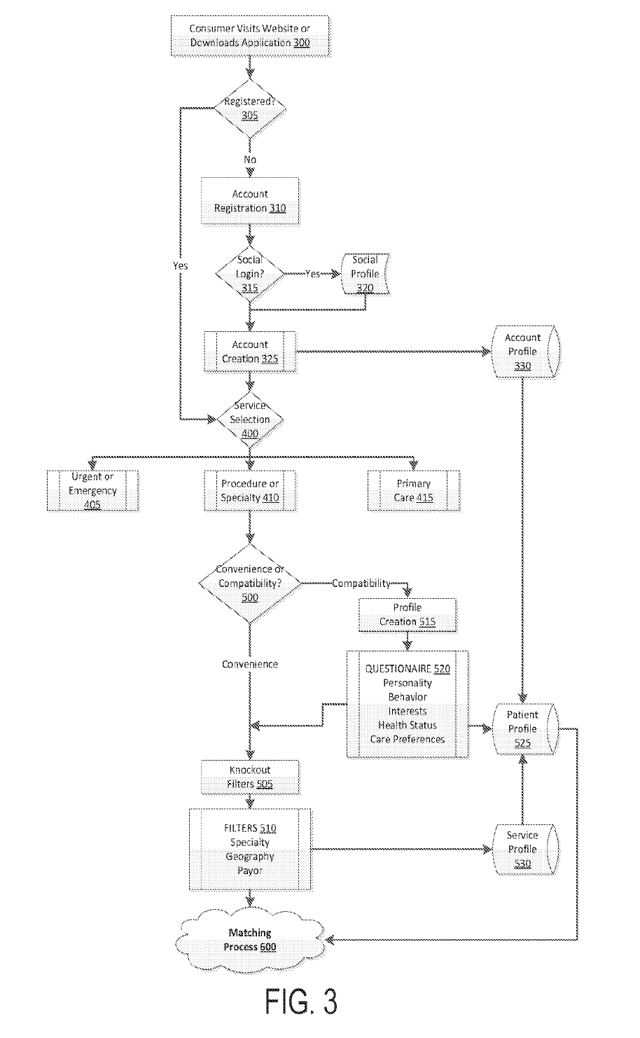

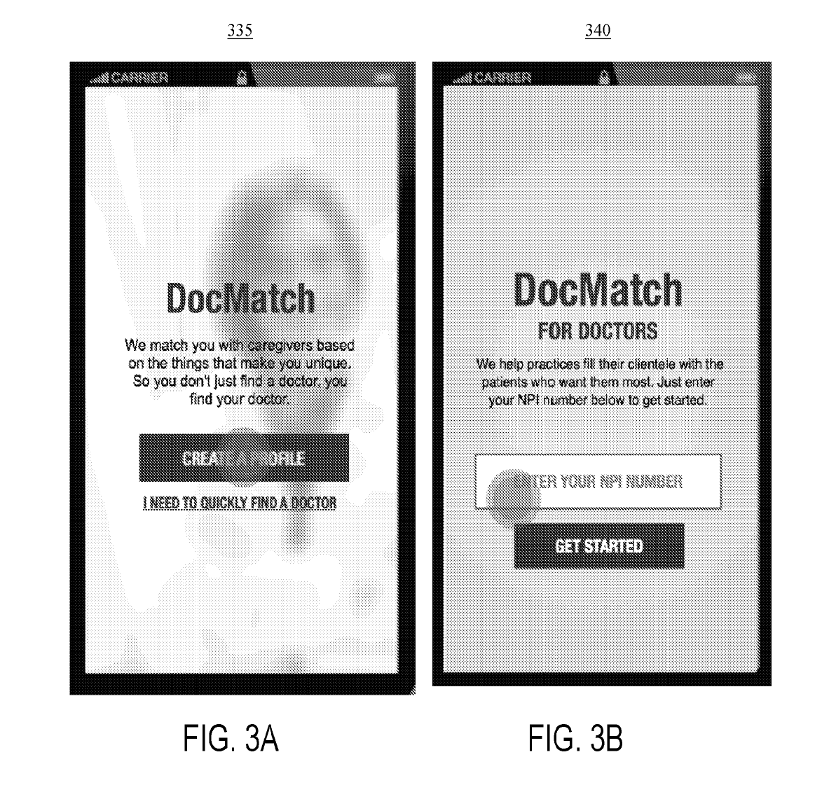

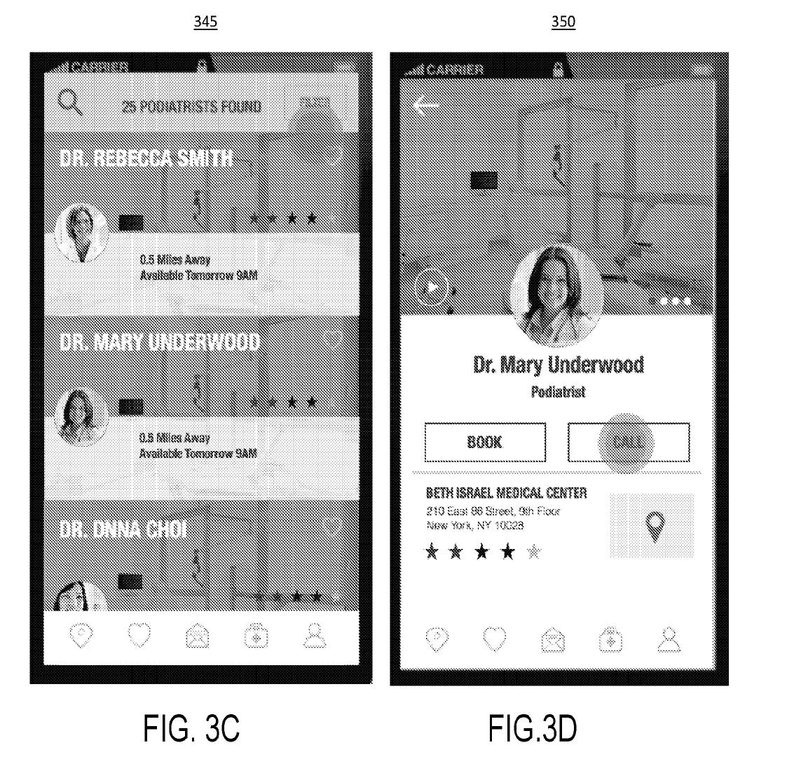

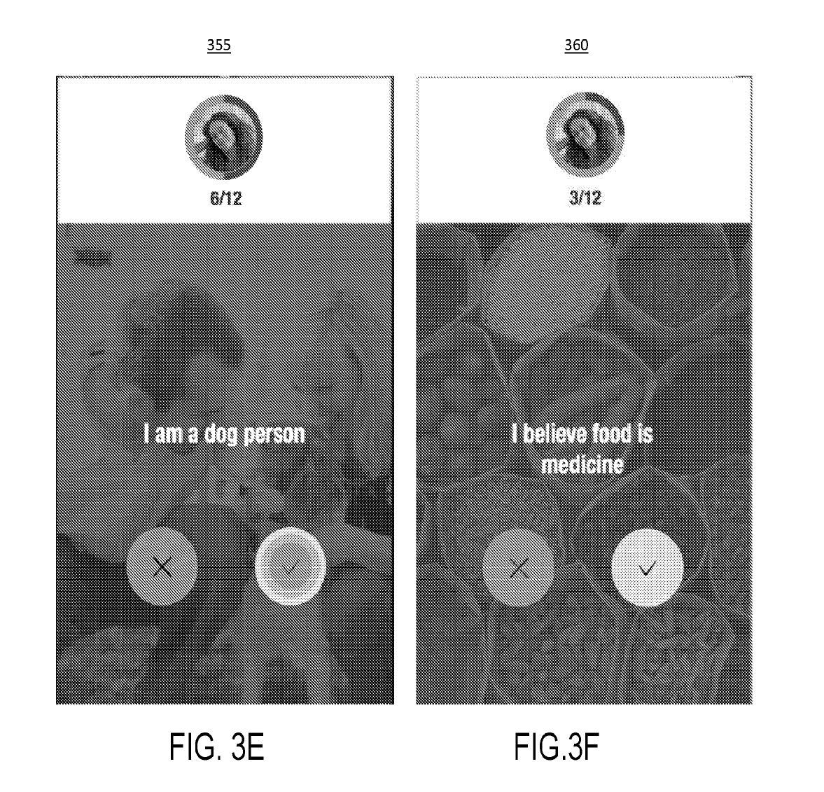

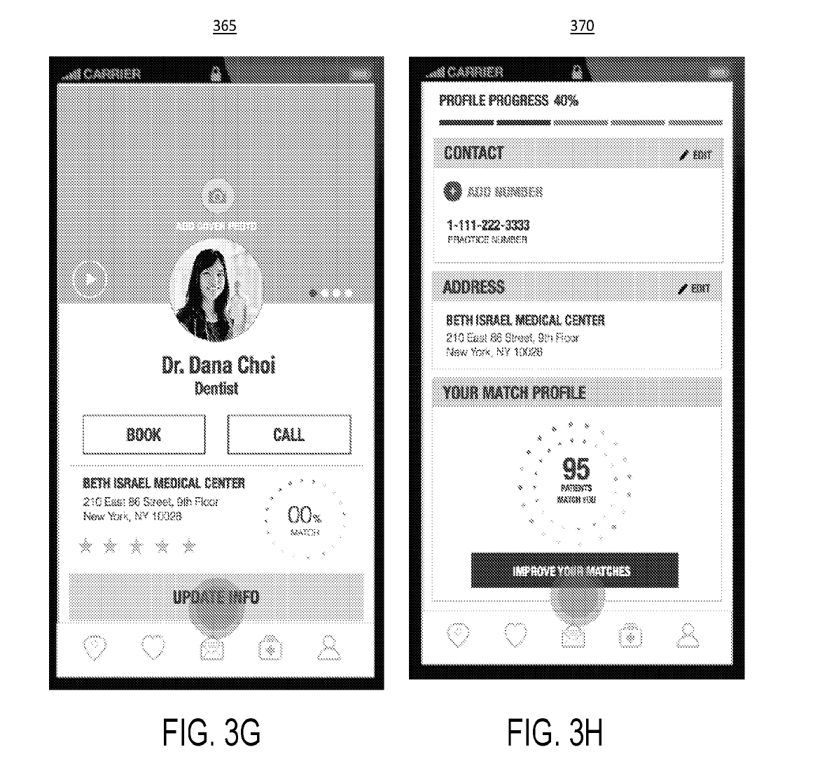

Returning now to FIG. 3, related flow charts and example user interfaces 102 setting forth more detailed exemplary steps for creating an account profile 330, a patient profile 525, and/or a service profile 530 are provided. In process block 300 of FIG. 3, a user, such as a patient, provider or consumer, may access the user interface generated by the GUI module 112 described above, and may further include the web page on a website viewed via a browser or may include a software application downloaded, installed and/or displayed on a client computer.

FIGS. 3A and 3B demonstrate non-limiting examples of the type of application that may be downloaded by the user including an interface to perform an account registration. As seen in FIGS. 3A and 3B, health care providers/physicians (referred to as providers herein) may also be provided a web page and/or download a software application to create an account. It should be noted that for each patient profile creation step disclosed throughout this disclosure, a complimentary or analogous provider profile creation step may exist, as discussed in more detail below.

Logic within the software modules and/or server computer(s) may receive user input indicating the user's access to the application. In decision block 305, the server computer(s) may log the user's access and determine, from any user input data associated with this access, if the user is already a registered user of the disclosed system. For example, the server computer(s) may query database 108 to determine if any account data records, associated with the received user input data, exist in database 108. If not, in process blocks 310 and 325, the software logic may create and/or register an account for a user, using techniques described herein.

In decision block 315, the server computer(s) may query database 108 to determine if database 108 stores data associated with one or more social media accounts for the user. If so, and the social media account data includes social media account login information, the server computer(s) may access an application programming interface (API) for the social media account in order to access a third party data feed for downloading, analyzing and storing the user's social media profiles and behaviors and adding them to user profile data records in database 108, as described in more detail below.

The account for the user may be created in process block 325, and the account details may be stored within an account profile 330, possibly comprising one or more data records stored in association with the user creating the account. This account and account profile may ultimately be added to the patient profile data 525, described in more detail below.

Once the user's account and account profile are established, (or if the server computer(s) determine in decision block 305 that the user already has an account and/or account profile), the user may input, possibly using one of the disclosed user interfaces, a selection of the type of medical services and/or treatment they currently need. In a non-limiting example embodiment, the user may select an urgent or emergency treatment, seen in process block 405, a procedure or specialty treatment, seen in process block 410, or a primary care treatment, seen in process block 415.

In decision block 500, the user may be presented a user interface with one or more user interface controls allowing the user to select between a need for convenience and accessing a series of user interfaces to determine compatibility between the user according to the patient's profile stored in database 108, and one or more physicians registered with the system according to one or more physician profiles stored in database 108. Using, the example interface in FIG. 3A, a user may select between creating a profile to determine the compatibility between the patient and one or more physicians, or an emergency situation, where the patient needs to quickly find a doctor without creating a profile.

Returning to decision block 500, if the user selects convenience, the server computer(s) may apply a series of filters limited only to the user's health utility needs, in order to quickly determine a match between the patient profile and one or more physician profiles stored within database 108, according to the matching algorithms disclosed herein.

A first non-limiting example may help to illustrate a patient that would choose convenience over compatibility. If a new system user, Brian, injures his ankle, and is on vacation without access to his doctor, he may create an account as described above, and access the landing page shown in FIG. 3A. However, rather than selecting the option to create a profile, as shown in FIG. 3A, Brian may select the option “I NEED TO QUICKLY FIND A DOCTOR,” thereby selecting convenience over compatibility, as seen in decision block 500.

The series of filters limited to health utility needs may include user preferences, either input as responses to questions presented through a user interface 102 or imported into database 108 from an API for the user's electronic health records as described below. In one non-limiting example, such as that shown in process block 510, the user preferences may include health utility needs information from the user, focused on details of the user's medical care. As non-limiting examples, health utility needs may include how many patients have seen a specific physician within a specific time period (e.g., the last year), how many patients highly ranked the physician, a physician's specialty (e.g., PCP, pediatrician, neurologist, orthopedic surgeon, etc.), a physician's location, a physician location range from a user (e.g., within 5 miles), accepting new patients (e.g., yes), experience with a particular medical procedure (e.g., radiofrequency ablation, MAZE procedure, etc.), experience treating a particular medical condition (e.g., atrial fibrillation, bradycardia, tachycardia, etc.), experience treating particular symptoms, experience treating patients of a particular age (e.g., over 50), experience treating patients of a particular gender (e.g., females), group practice (e.g., PAMF), education (e.g., degrees, names of institutions, locations, years of graduations, ranking within institutions), credentials (e.g., board certifications, special awards, honors), gender (e.g., female or male), languages spoken (e.g., English & Spanish), office hours (e.g., after 5 PM), and a physician's age (e.g., less than 60). Other preference data specified at process block 202 may include a preferred subspecialty (e.g., pediatric EarNose-Throat (ENT), pediatric behavioral disorders, etc.), experience details (e.g., name of procedures performed, name of conditions treated, treated patients ages and genders), and further educational details (e.g., medical schools by rank, Ph.D., j.D., etc.); whether the user's payor, such as an the user's insurance company, is compatible between the user and the selected physician, etc.

Additionally or alternatively, at process block 202, the physician recommendation system 100 may prompt the user with specific questions regarding their preferences for a physician and provide responses to be used as recommendation criteria. In some embodiments, the data collected from the user may be stored within a service profile 530.

Returning to the first example with user Brian, after creating an account and selecting between convenience and compatibility, Brian's client device may allow Brian to input insurance data, desired medical specialty data and/or location data, as non-limiting examples. In some embodiments, Brian's insurance, specialty and/or location data may be stored within a service profile 530.

Using the input user data relating to the user's health utility needs, the server computer(s) may proceed to the user/physician matching process in process block 600. Using Brian as an example, the server computer(s) may receive Brian's input data relating to his health utility needs and access database 108 to determine physician profiles that match those health utility needs. These physician profiles may have been previously established using the method steps described in more detail below. The server computer(s) may then render a user interface similar to that seen in FIG. 3C, listing physicians having profiles that match Brian's health needs. The user may then refine the filters using a user interface according to other desired parameters, such as availability, location, language, gender, etc. The server computer(s) may then match the user's refined filters to one or more matching physician profiles in database 108.

Returning to decision block 500, if the user selects determining a compatibility between the user's profile and one or more physician profiles (i.e., “CREATE A PROFILE” in FIG. 3A), the server computer(s) may create a patient profile at process block 515 (if not previously created) and store user profile data 525, possibly as a series of patient profile data records, in database 108.

Profile data 525 may also be obtained at process block 202 and may include, for example, patient name, address, additional contact information (address, telephone number, email, texting preferences, etc.), family information, healthcare information, and information about a particular physician associated with the user (e.g., physician's name, physician's address, physician's practice area, etc.). In one example, the profile data may also include user comments and/or ratings on experiences with a particular physician that can later be used by the recommendation module 106 to recommend higher ranked physicians to patients having similar preferences.

The profile data 525 may also be obtained from a questionnaire 520 generated within a user interface, which receives input data from the user associated with the profile. In process block 515, once the patient profile is created, the server computer(s) may generate one or more user interfaces to present the user with a questionnaire to determine additional patient profile data 525 regarding, for example, the patient's personality, behavior, interests, health status, care preferences, etc.

Each of the questions within the questionnaire may define an attribute for the user, and each question may lie associated with a particular category for the attribute.

The user may input their responses, possibly including a value and weight for each response, into the user interface. These responses may then be stored in database 108, possibly as patient profile data records 525. As non-limiting examples, the questionnaire may include the responses to questions within the questionnaire, broken down by categories such as personality, behavior, interests, health care status, care preferences, etc., as well as the value, weight and/or predictability of the response. For example:

TABLE 1
Questionnaire Responses for Questions in the Health Care Preferences
Category
Health Care Preferences

| Question | Value | Weight | Predictability |
|---|---|---|---|
| I'm loyal to my doctor(s) | 0 | 5 | High |
| I expect my Dr to manage my care | 1 | 5 | Med |
| I expect my doctor to lead my care plan | 0 | 5 | High |
| I expect my doctor to tell me what to do | 1 | 5 | High |
| I don't typically trust doctors | 0 | 5 | Med |
| I value a second opinion | 1 | 5 | High |
| If I'm not comfortable with my | 0 | 5 | Low |
| diagnosis, I will see a different doctor | | | |
| I like to research my health and | 1 | 5 | High |
| healthcare online | | | |
| I tend to self-diagnose my health issues | 1 | 5 | High |
| I'm less concerned with seeing “my” | 1 | 5 | Med |
| doctor if it means I can be seen when I | | | |
| want to be seen | | | |
| I want a doctor that communicates | 0 | 5 | Low |
| electronically | | | |
| I want t doctor that I can schedule my | 0 | 5 | High |
| appointments online | | | |
| I'm interested in alternative medicine | 1 | 5 | Med |
| I expect my doctor to treat my mind, | 1 | 5 | Med |
| body and spirit | | | |

TABLE 2
Questionnaire Responses for Questions in the Personality Category
Personality

| Question | Value | Weight | Predictability |
|---|---|---|---|
| I'm a reserved person | 0 | 3 | Med |
| I act on emotion | 0 | 3 | Low |
| I find the best in others | 1 | 3 | Low |
| I need positive feedback | 1 | 3 | High |
| I try to make the world around me a | 0 | 3 | Med |
| better place | | | |
| I'm a good listener | 1 | 3 | High |
| I avoid conflict | 0 | 3 | High |
| I'm always looking for ways to improve | 1 | 3 | High |
| things | | | |
| I have a hard time expressing my | 1 | 3 | Med |
| feelings and emotions verbally | | | |
| I value order and structure | 1 | 3 | Med |
| I always try to bring out the best in | 0 | 3 | Low |
| others | | | |
| I am an extrovert | 0 | 3 | High |

As seen in the tables above, each data record may also include a weight assigned by the user to the user's perceived importance of the attribute. In some embodiments, the user may input, with the response to each question, a numeric value or range of values representing the weight that should be assigned to the attribute. Each of the data records may also include a predictability weighting. This weighting may be derived from analysis of user feedback data (e.g., a post doctor appointment survey rendered and transmitted to a user client device via email, text, etc.). This user feedback data may be aggregated from multiple users in order to track the accuracy and effectiveness of the patient profile/physician profile match, discussed in more detail below. Weighting may also occur based on the predictive nature of the attribute. In other words, certain attributes will receive a higher weight if they represent attributes

In another example, user typographical errors may be corrected when errors are made within fields on the interface 102 that require manual entry. For example, if a user misspells a medical term or a name, a user is prompted whether he/she really meant something, else (i.e., a medical term that may have the correct spelling), and submits the preferences to the recommendation module 106 according to the user's response.

In another example embodiment, the interface 102 may provide the user with an application program interface to receive third party data feeds which may be downloaded and stored in the database 108 in association with the patient and/or provider profile account. This downloaded data may be parsed, tokenized and/or analyzed to supplement the questionnaire response data according to a category and/or attribute assigned to each data received from, for example, a social media account and/or medical data database. To access the API, the user may provide authentication information in order to access, download and/or store the user's data in database 108. In this way, patients may append their profiles by connecting their social media profiles or electronic health records to their patient or provider profile. Data from these third party providers may auto-populate select profile questions/categories.

For example, the provided software may access the user's social media accounts via the API, that is configured to acquire the user's preference data from a social network, such as Facebook. For example, user preference data may be determined based on the user's Facebook profile information (e.g., name, address, phone number, etc.). Additionally, the user preference data may be determined based on the user's Facebook likes, what people and/or organizations the user follows on Facebook, types of websites that the user shares on Facebook, the content of the user's comments on Facebook, and the like.

For example, if a user likes a particular university on Facebook (e.g., University of Michigan), this may be used by the recommendation module 106 to match the user with a physician who graduated from or is affiliated with the particular university. In another example, if the user shares a website and/or article on Facebook related to the promotion, of certain types of vaccinations, this data may be used by the recommendation module 106 to match the user with a physician who indicates similar beliefs on certain vaccinations. In yet another example, if the user posts one or more comments on Facebook related to a positive experience at a particular healthcare facility, the recommendation module 106 may use this data to match the user and other users with a physician who practices at the particular healthcare facility.

Similar user preference data may be acquired from other social networks including, Twitter, Linkedin, and the like. User preference data may also be obtained from the user's electronic medical record or a database of user profile data. The various user preferences acquired from the social network may be tracked and stored in the shared database 108 and utilized by the recommendation module 106 to recommend one or more physicians whose characteristics are in-line with the user preferences. Similarly, the one or more software modules may connect to an API, after authentication, for one or more sources, such as Medicare Blue Button, as a non limiting example, for the user's electronic health records.

A second non-limiting example may help to illustrate a patient that would choose compatibility over convenience. If a new system user Elena is looking for a pediatrician for her daughter Vicky (who has AMID), that is close to home, communicates via email and text messaging, and share's Vicky's interest in sports, Elena may create an account as described above, and access the landing page shown in FIG. 3A. As shown, Elena may select the option to “CREATE A PROFILE,” thereby selecting compatibility over convenience, as seen in decision block 500.

Elena may then customize her profile by accessing a questionnaire generated by the server computer(s) and transmitted to and displayed on her client device. FIGS. 3E-3F are non-limiting examples of the types of questionnaire questions that may be presented to a user such as Elena.

The feedback from user questions may be provided using any form of receiving user feedback from a user interface. For example, the user may respond to questions using the positive or negative responses shown in FIGS. 3E-3F. In some embodiments, the user interface may include a slider representing a continuum with two opposing values on opposite ends (i.e., a positive response at one end, and a negative at the other). For example, a user may select between a friendly doctor and a serious doctor. The amount of the slide may represent a weight of importance the user is assigning to the user preference.

Provider profiles may be created (analogous to process block 515) and stored (analogous to process block 525) in a similar manner and the stored provider profile data 525 and service profile data 530 may be analogous to those stored for the patient profile data 525 and service data 530. The physician data for each of the physicians in the database may be obtained through a user interface presented to the physician using a process similar to that of the patient seen in FIGS. 3A-3H.

A third non-limiting example may help to illustrate a provider/physician creating a provider/physician profile. If a new system user and dentist, Dana, is looking to find an easier way to connect with her patients and make them feel more involved with their care, as well as build her office clientele within the Chinese-American community, whom she shares philosophies with, Elena may create an account as described above, and access the landing page shown in FIG. 3B. As shown, Elena may enter her NPI number and select the “GET STARTED” button to access the provider/physician profile setup. Using the NPI number, the server computer(s) may access third-party APIs and other platforms to quickly pull her professional information into her provider/physician profile.

By analogy to the patient profile processes in FIGS. 3A-3F, the provider/physician may enter data, or have data imported using third party APIs or other platforms to enter data about the services provided, which may be complimentary to the classification of service (e.g., Urgent or Emergency 405, Procedure or Specialty 410, or Primary Care 415) that patients will enter for their patient profile. Similarly, data may be received and stored for the filters data in process block 510 that will be compared with the input data that patients will enter when setting up their patient profile.

Thus, after creating her account, Dana may then be presented with a provider profile template 700 as seen in FIG. 3G. By selecting the “UPDATE INFO” button in this example, she may edit her profile to enter any missing information about her practice, specialties, credentials, etc., that were not added through her NPI number. In addition, Dana may add or update additional profile information such as schools she's attended and languages she speaks, interests, etc. Dana may also add pictures or other graphics to her profile. Based on her profile to that point, the server computer(s) may render and display a user interface 370, such as that seen in FIG. 3HI displaying, her progress, including the number of user profiles in database 108 that match her profile, and a link to improve her matches.

Providers may therefore further customize their profile and improve the number of matching profiles by answering a questionnaire analogous and/or complimentary to the patient questionnaire in process block 520, as seen in FIGS. 3E and 3F. As with the patient questionnaire, each question and response may be associated with a category and/or attribute data. The providers/physicians may then finalize their profile data and publish a website.

Both the user preference data, the associated category and attribute data, and profile data obtained at process block 202 may be stored in the database 108, as well as physician related data, for example, that is accessible by the recommendation module 106 to match the user data with the physician data to recommend one or more physicians to the patient. In order to protect the patient's personal information, an authentication service may be configured to ensure that only authorized users are given access to the physician recommendation system 100. For example, the authentication service may require the user to present a username and/or password, an encrypted digital signature, or any other type of authorization credential recognized as valid by the authentication service. In one or more embodiments, the database 108 may be located in a local area network (LAN) and the authentication service includes a firewall protecting the LAN from unauthorized access.

Once all data is gathered from each provider, and the user preference, response, category, attribute, weighting and/or profile data are obtained at process block 202, the recommendation module 106 may be configured to compare the user preference data and physician data to identify potential physicians at process block 204. The physicians identified at process block 204 may satisfy one or more of the user preferences identified at process block 204. The recommendation module 106 is able to crawl the user data and physician data stored in the database 108 to compare the data and identify potential physicians for the user.

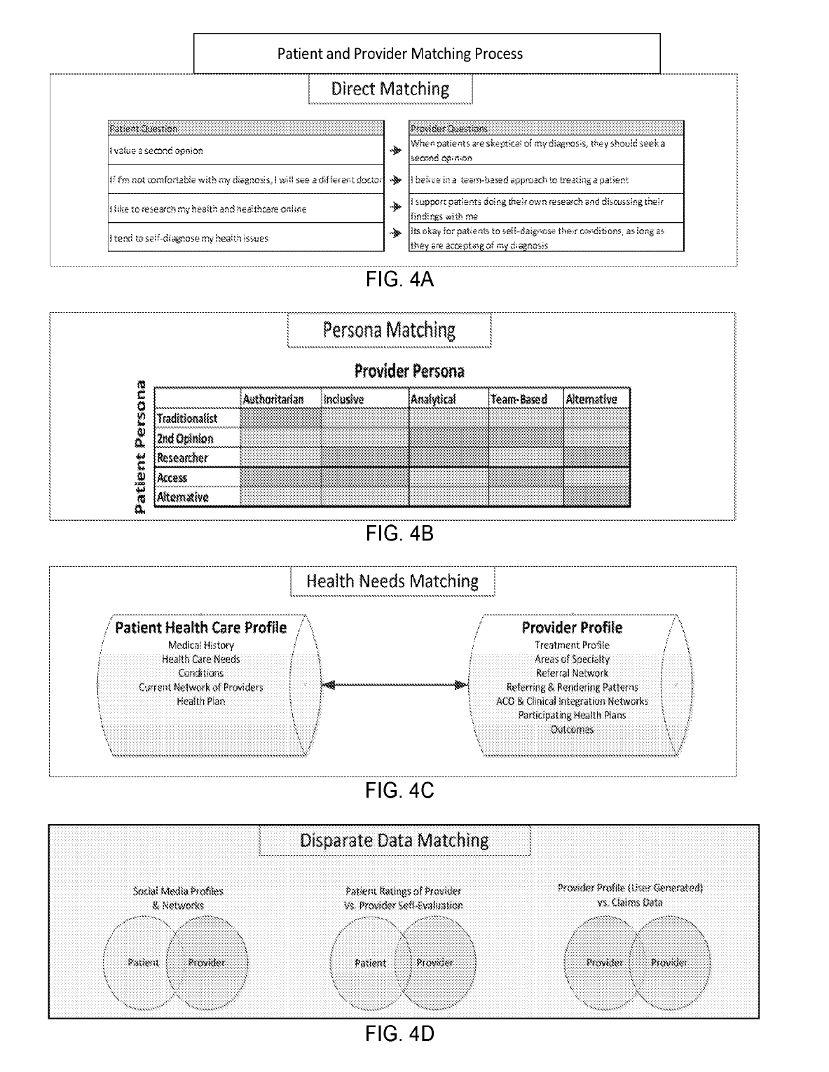

A first way the data between patients and providers may be matched is by direct matching, as seen in FIG. 4A. Certain questions within a patient's profile have a direct correlation to related questions within a provider's profile. Matches are determined based on a one to one match between the correlated question sets. Additional weighting is applied to matched/unmatched attributes based on two factors: 1. Those matches determined to produce more favorable health care outcomes are weighted more heavily; and 2. Patients have the opportunity to prioritize certain compatibility attributes as having more relative importance to their health care experience.

A second way the data between patients and providers may be matched by persona matching, as seen in FIG. 4B. Based on their responses to questions within the software, patients and providers are both mapped to either a single person or are identified as having tendencies more closely related to up to two personas. Patient personas are then aligned with compatible physician personas during the matching process.

A third way the data between patients and providers may be matched is by health needs matching, as seen in FIG. 4C. Health needs matching compares data elements from a patient's health care profile (e.g., medical conditions, health care needs, health plan, etc.) with related elements from a provider's profile, in order to more effectively match patients with providers that are: 1. More compatible with other “like” patients; and 2. Who have the likelihood of producing more favorable outcomes (e.g., satisfaction, adherence, outcomes, preference).

A fourth way the data between patients and physicians may be matched is by disparate data (behavioral) matching, as seen in FIG. 4D. Disparate data matching associates profile attributes from different data sources, to more effectively validate user-generated data with third party data. This method of matching helps to validate user generated profile data and confirm the validity of the profile claims. Matched data elements include health care practice data for providers using claims data, hobbies/interest matches between patients and providers via social media profiles, and provider practice/personality attributes based on patient reviews, discussed below.

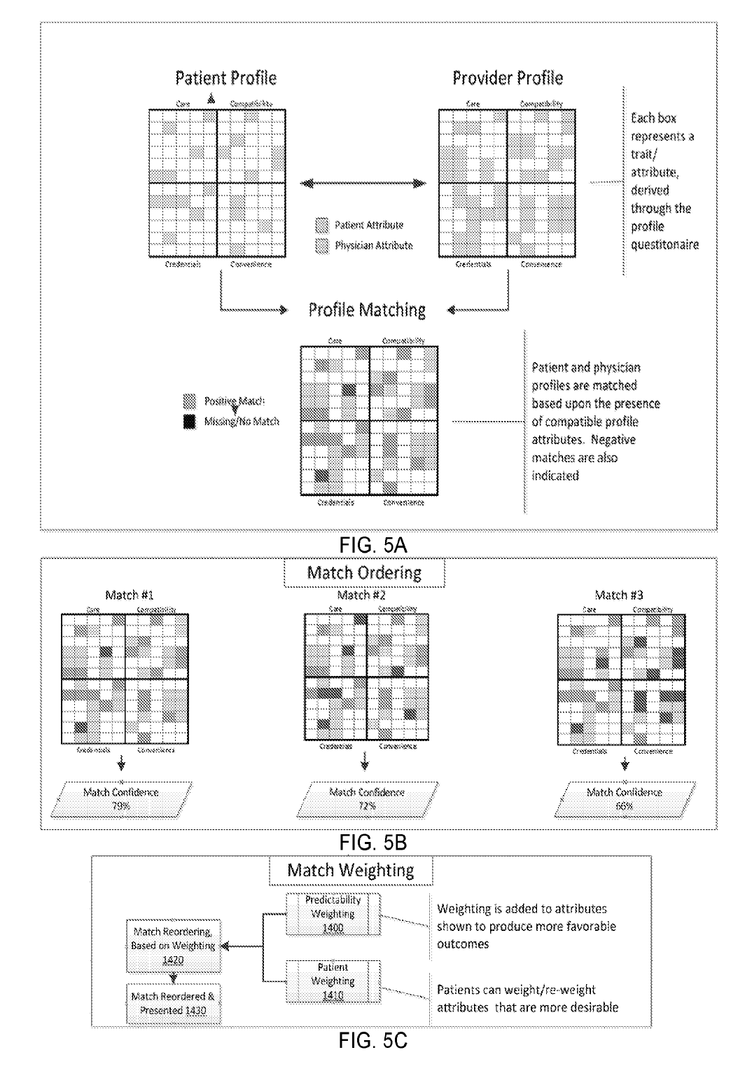

FIG. 5A is a visualization used to show the overlapping individual traits/attributes (e.g., attributes where the patient profile attribute and provider profile attribute are common), derived through the patient and provider profile questionnaires, or other matching techniques described above. These attributes may be analyzed and matched according to the presence of compatible profile attributes. A positive match of these compatible attributes (e.g., those attributes with overlapping attributes) are indicated by the lighter squares in FIG. 5A and a negative or non-match is indicated by the darker squares.

In this non-limiting example, the match is based on responses to questions in the questionnaire, each of the questions/responses representing a user attribute, each of the questions/responses falling into one of four quadrants, and each of the quadrants representing a category associated with each of the questions/responses. In the example embodiments in FIG. 5A, the quadrants/categories may include a provider's/patient's preferred care, compatibility, credentials and convenience.

FIG. 5B demonstrates how the server computer(s) calculate a match confidence percentage between the patient attributes for each of the categories and the provider attributes for each of the categories. In these examples, the number of overlapping attributes represents the percentage of match confidence for each of the four categories, and as a whole. The match confidence percentage then determines whether providers are a match above the threshold and the order that matching providers are presented to the user.

As noted above, and demonstrated in FIG. 5C, in some embodiments, the match may be weighted according to attributes shown to produce more favorable outcomes, as demonstrated in process block 560. The weights for these attributes may be determined based on analysis of follow up and prescription results from user feedback, as described below. The weights for these attributes may also be based on weights that each patient has placed on attributes that the patient has designated as more desirable, as demonstrated in process block 565

After running the match ordering analysis, the one or more software modules may then rerun the match ordering algorithm, but weight the match confidence level according to the weighting of the attributes shown to show more favorable results and/or those attributes weighted as more desirable by the patient, as demonstrated in process block 570. In process block 580, the match may then be reordered according to any weighting, and may then be presented to the user.

Next, returning to FIG. 2, at decision block 206, the recommendation module 106 determines whether each of the potential physicians identified at process block 204 is above a predetermined threshold. The threshold may be, for example, a percentage valve (e.g., 80%) indicating a percentage of the user preference data that matches the physician data. Thus, if 85% of the user preference data matches the physician data, that physician is above the threshold at decision block 206. However, if only 70%, for example, of the user preference data matches the physician data, that physician is below the threshold at decision block 206, and will not be recommended. Thus, the recommendation module 106 will continue to obtain user preferences and profile data at process block 202 until the threshold is met.

The potential physicians that meet or exceed the threshold at decision block 206 may then be ranked at process block 208. For example, the potential physicians may be ranked according to the number of times a particular physician has been saved as a favorite physician. Therefore, the highest ranking physician would be the physician that is the most popular among patients or potential patients. Another method of ranking physicians involves filtering recommendations based on users with common attributes. Thus, the highest ranking physician would be one that has been saved as a favorite physician by other users with similar demographic characteristics as a current user searching for a physician. Yet another ranking methodology may include ranking physicians based on the number of times that they are chosen as potential physicians by a user. Additionally, rankings may be based on the number of times a physician has actually been scheduled for appointments with users.

Additionally, or alternatively, the physicians may be ranked according to physician feedback that is input into the physician recommendation system 100 by different users. The input may be based on previous experiences with a specific physician, as described in more detail below, and may reveal both positive and negative aspects of those experiences (e.g., physician was very easy to work with, physician did not discuss concerns of patient sufficiently, etc.). The experiences may relate to a specific medical condition, or may be very broad in nature.

After ranking the physicians at process block 208, the recommendation module 106 may display a list of recommended physicians to the user at process block 210. The list of recommended physicians may be displayed on the interface 102, for example, with the highest ranking physician as the first entry, and the lowest ranking physician as the last entry. The display of the list of recommended physicians may include the user preference information (e.g., group practice) and the physician's photograph, for example.

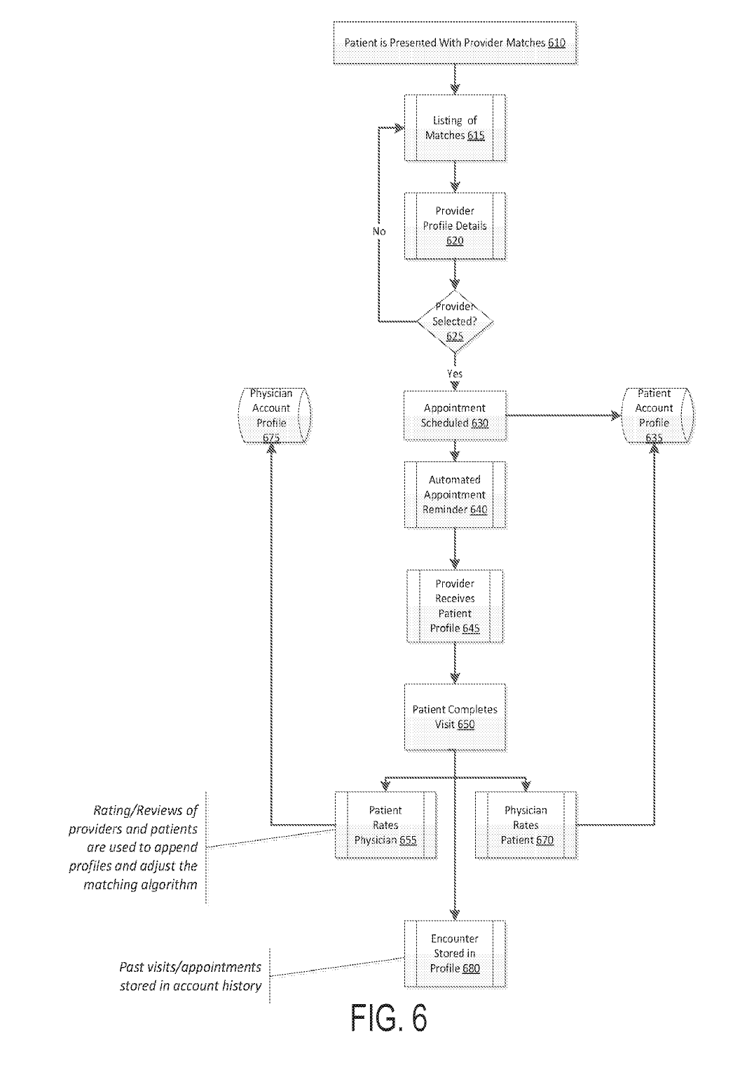

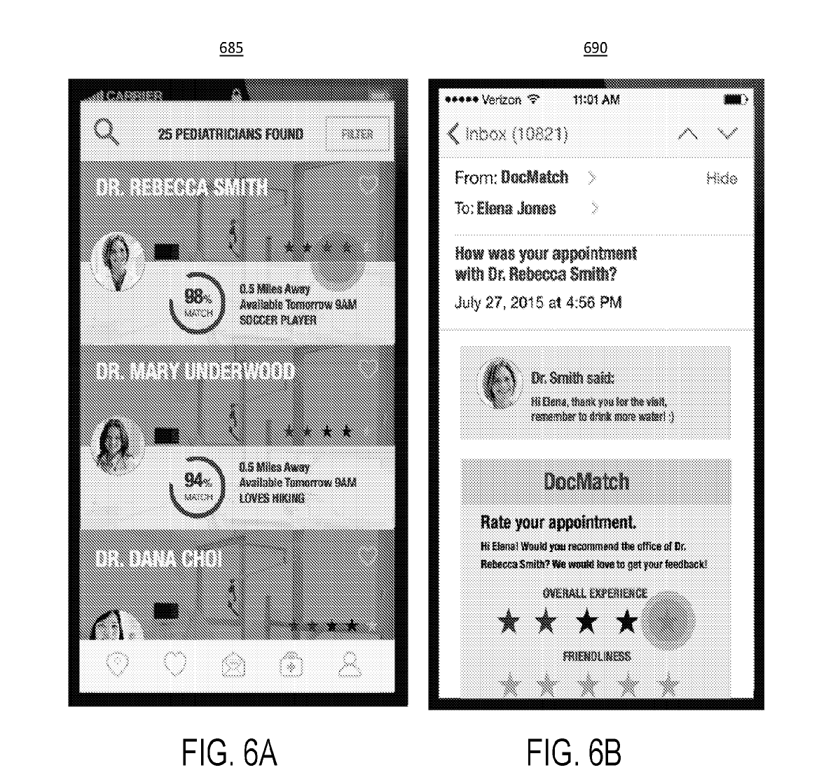

Referring now to the more detailed flow chart in FIG. 6, the server computer(s) may render a user interface to present the user with a listing of provider matches in process blocks 610 and 615, possibly in order of the ranking established above. In some embodiments, the displayed list of recommended physicians may be limited to a manageable number of items per page (e.g., 5), with GUI navigation to previous and next pages. FIG. 6A demonstrates a list of providers/physicians presented to a user, specifically Elena from the example above, after her profile was matched to providers within database 108.

In process block 620, each of the matches may include means, such as a hyperlink to a web page or other additional user interface, to access additional details for the physician. These details may be stored as part of the physician profile in database 108. A web page link, quick view or rollover feature may enable more information when a user performs a mouse-over on a physician's name or photograph, in the form of a pop up window with additional details. The quick view may include a physician's information including personal statement, address, phone, URL of the physician's website, office hours, and a link to a map of the location of his/her office, as non-limiting examples.

In some embodiments, the recommendation module 106 may provide the user with a comparison tool at process block 212. The comparison tool allows the patient to compare two or more physicians in a side-by-side view, for example. The user may review information that is likely to help him/her decide between physicians (e.g., experience, subspecialty, photo, physician's personal statement, location, and office hours), as well as information regarding, appointments. After comparing physicians' profiles, the user may select a physician from the list of recommended physicians at process block 214.

Returning now to FIG. 6, in decision block 625, the server computer(s) may determine whether a provider has been selected. Once the user selects a physician at process block 214, and once the server computer(s) determine that the provider has been selected in decision block 625, the user may schedule an appointment with the physician, or the server computer(s) may automatically schedule an appointment, as seen in process block 630. Returning to the Elena example, after Elena select Dr. Rebecca Smith, based on her 98% match, Elena may schedule an appointment or learn more about Dr. Smith.

The physician and the patient may each receive a download of the other's respective background through their profile, as seen in process block 645, in advance of the visit so they are familiar with each other prior to the visit. The patient may complete the visit in process block 650, and a feedback module may be provided on the interface 102 that prompts the user to provide feedback related to the list of recommended physicians at process block 216. For example, a survey may be provided to determine whether the user thought the list of recommended physicians met the user preferences previously provided. FIG. 6B is a non-limiting example interface 690, used by Elena to rate Dr. Smith after her appointment.

Based on the user's feedback, the physician recommendation system 100 may update the recommendation module 106 to continuously provide accurate lists of recommended physicians for the user. The recommendation module may accomplish this through use of the feedback provided by both the patient and the provider after the visit. As each patient leaves feedback rating and reviewing the provider, as seen in process block 655, the physician profile 675 may be updated with this supplemental information. Similarly, the provider may rate the patient in process block 670, and the patient's account profile 635 may be updated accordingly.

Any provider feedback provided by users may be stored as feedback data in database 108. The server computer(s) may analyze this provider feedback to generate and store a plurality of factors used to determined the greatest predictability for future positive experiences by all users and patients. The analysis may comprise a statistical analysis of highest ranking prioritized factors that resulted in the highest positive feedback from the users after their scheduled appointments.

With an established account, the patient may then provide supplemental information, such as scheduling follow up appointments, or moving from a long term care provider relationship to a specialist. For example, if the patient needed a cardiologist due to a bad heart valve, they can specify the procedure needed, and the type of doctor (cardiologist). By asking 2-3 supplemental questions specific to the health condition, they can supplement the profile that already defines who the user is. The user account may therefore be continually evolving to provide the best possible matches for the customer's needs.

The present invention has been described in terms of one or more preferred embodiments, and it should be appreciated that many equivalents, alternatives, variations, and modifications, aside from those expressly stated, are possible and within the scope of the invention.

1. A system comprising a processor executing instructions causing a server computer, coupled to an electronic network, to:
receive, through the electronic network a transmission encoding:
a plurality of patient attribute data defining a patient preference profile; and
a request to match the plurality of patient attribute data with a plurality of physician attribute data;
execute a first database query to store the plurality of patient attribute data in a database coupled to the electronic network;
execute a second database query to identify the plurality of physician attribute data storing a plurality of common attribute data with the plurality of patient attribute data, the common attribute data being stored within a plurality of data fields comprising:
a category data defining, a preference type category for the common attribute data;
a sentiment data defining a positive or negative sentiment about the common attribute data;
a patient preference weight value defining a weight to be applied to the category data and the sentiment data; and
a predictability weight value defining, a likelihood of a positive outcome from the common attribute data;
render a user interface recommending a list of physicians associated with the common attribute data; and
transmit, through the electronic network, the user interface to the client computer for display.

2. The system of claim 1, wherein the plurality of patient attribute data is stored in the database in association with a patient account, and the plurality of physician attribute data is stored in the database in association with a physician account.

3. The system of claim 1, wherein the plurality of common attribute data is limited to a plurality of data records defining a plurality of health utility needs.

4. The system of claim 1, wherein the plurality of common attribute data is filtered according to a plurality of data records defining a plurality of health utility needs.

5. The system of claim 1, wherein the plurality of patient attribute data and the plurality of physician attribute data are received using:
a questionnaire comprising a plurality of questions, each of the plurality of questions associated with a category and defining a patient attribute or a physician attribute; or
a profile data downloaded through the electronic network using an application programming interface and used to access a social network account or a medical data database.

6. The system of claim 1, wherein the plurality of common attribute data is identified by direct matching, persona matching, health needs matching, or disparate data matching.

7. The system of claim 1, wherein the instructions cause the server computer to:
receive a user input selecting a physician within the list of physicians; and
schedule an appointment with the physician.

8. The system of claim 7, wherein the instructions cause the server computer, subsequent to the appointment, to:
transmit a request for a user feedback related to the appointment; and
receive the user feedback.

9. The system of claim 8, wherein the predictability weight is determined by an aggregation of the user feedback for a plurality of users.

10. The system of claim 1, wherein the plurality of common attribute data is modeled against a plurality of historical health care outcomes data to identify a plurality of predictive attributes that, when present, correspond to a highest quality of a patient outcome.

11. A method comprising:
receiving, by, a server computer, through an electronic network, a transmission encoding:
a plurality of patient attribute data defining a patient preference profile; and
a request to match the plurality of patient attribute data with a plurality of physician attribute data;
executing, by the server computer, a first database query to store the plurality of patient attribute data in a database coupled to the electronic network;
executing, by the server computer, a second database query to identify the plurality of physician attribute data storing a plurality of common attribute data with the plurality of patient attribute data, the common attribute data being stored within a plurality of data fields comprising:
a category data defining a preference type category for the common attribute data;
a sentiment data defining a positive or negative sentiment about the common attribute data;
a patient preference weight value defining a weight to be applied to the category data and the sentiment data; and
a predictability weight value defining a likelihood of a positive outcome from the common attribute data;
rendering, by the server computer, a user interface recommending a list of physicians associated with the common attribute data; and
transmitting, by the server computer, through the electronic network, the user interface to the client computer for display.

12. The method of claim 11, further comprising the step of storing, by the server computer, the plurality of patient attribute data in the database in association with a patient account, and the plurality of physician attribute data in association with a physician account.

13. The method of claim 11, wherein the plurality of common attribute data is limited to a plurality of data records defining a plurality of health utility needs.

14. The method of claim 11, further comprising the step of filtering, by the server computer, the plurality of common attribute data according to a plurality of data records defining a plurality of health utility needs.

15. The method of claim 11, further comprising the steps of receiving, by the server computer, the plurality of patient attribute data and the plurality of physician attribute data using:
a questionnaire comprising a plurality of questions, each of the plurality of questions associated with a category and defining a patient attribute or a physician attribute; or
a profile data downloaded through the electronic network using, an application programming interface and used to access a social network account or a medical data database.

16. The method of claim 11, further comprising the step of identifying, by the server computer, the plurality of common attribute data by direct matching, persona matching, health needs matching, or disparate data matching.

17. The method of claim 11 further comprising the steps of:
receiving, by the server computer, a user input selecting a physician within the list of physicians; and
scheduling, by the server computer, an appointment with the physician.

18. The method of claim 17, further comprising the steps, subsequent to the appointment, of:
transmitting, by the server computer, a request for a user feedback related to the appointment; and
receiving, by the server computer, the user feedback.

19. The method of claim 18, further comprising the step of determining, by the server computer, the predictability weight according to an aggregation of the user feedback for a plurality of users.

20. The method of claim 11, further comprising the step of modeling, by the server computer, the plurality of common attribute data against a plurality of historical health care outcomes data to identify a plurality of predictive attributes that, when present, correspond to a highest quality of a patient outcome.
